(19) United States
(12) Patent Application Publication
(10) Pub. No.: US 2010/0068291 A1
(43) Pub. Date: Mar. 18, 2010

(54) Oral Medicament Based on a Proton Pump Inhibitor

(75) Inventors: Philippe CAISSE, St-Bonnet-de-Mure (FR); Catherine CASTAN, Orlienas (FR); Rémi MEYRUEIX, Lyon (FR); Gérard SOULA, Meyzieu (FR)

Correspondence Address: PATTON BOGGS LLP, 8484 WESTPARK DRIVE, SUITE 900, MCLEAN, VA, 22102, US

(73) Assignee: FLAMEL TECHNOLOGIES, S.A., Venissieux Cedex (FR)

(21) Appl. No.: 11/920,278

(22) PCT Filed: May 15, 2006
(86) PCT No.: PCT/EP2006/062321

§ 371 Date: Mar. 9, 2009

(30) Foreign Application Priority Data
May 13, 2005 (FR) 05 51259

(51) Int. Cl. A61K 9/50; A61K 31/4439; A61K 31/426; A61K 31/4164; A61K 31/341; A61K 47/02; A61K 47/06

(52) U.S. Cl. 424/494; 424/490; 424/497; 514/338; 514/365; 514/370; 514/400; 514/471; 514/769; 514/770; 514/772

(57) ABSTRACT

The invention relates to oral medicaments having a modified release of proton pump inhibitors (PPI's) that are, in particular, useful in preventing and treating gastrointestinal disorders. The aim of the invention is to provide a novel oral medicament based on PPI's ideally having all or some of the following characteristics: a) quickly providing relief to the patient by increasing the gastric pH after oral administration of the medicament; b) accelerating the recovery of patients while maintaining this increase in the gastric pH for as long as possible after oral administration of the medicament and, in particular, during the night; c) improving the observance of the treatment and the comfort of the patient by taking the medicament once daily. To this end, the microcapsules of the invention, preferably non-enteric, are constituted of PPI microparticles coated with ethyl cellulose, an ammonio methacrylate copolymer (Eudragit® RL 100), polyvinylpyrrolidone, castor oil and polyoxyethylenated hydrogenated castor oil (40). This medicament is designed so that after its ingestion for a once daily administration, it makes it possible to maintain, from the first day of treatment onward, an average gastric pH, between 0 and 24 h, of greater than or equal to the average gastric pH between 0 and 24 h obtained by an enteric oral medicament having a reference* immediate release, administered under the same conditions. The invention also relates to these microcapsules per se.

### FIELD OF THE INVENTION

The field of the invention is that of medicaments that can more particularly be used for the prevention and treatment of gastrointestinal disorders.

The present invention relates to an oral medicament based on at least one proton pump inhibitor (PPI) and allowing modified release of the PPI.

By convention, the acronym “PPI” used in the singular in the present disclosure will denote without distinction one or more PPIs, with the exclusion of lansoprazole and/or of at least one of the lansoprazole metabolites.

More specifically, the oral medicament according to the invention preferably comprises a plurality of microcapsules of PPI, in each of which the PPI is contained in a microparticle which is itself covered with a coating for modified release of the PPI in the gastrointestinal tract or under corresponding in vitro conditions.

The invention also relates to the PPI microcapsules taken as such.

In the present disclosure, the “PPI” active ingredient denotes without distinction one or more PPIs per se and/or one or more PPI metabolites and/or one or more PPI derivatives and/or any mixture of these active agents.

### PROBLEM AND PRIOR ART

PPIs are inhibitors of gastric acid secretions through specific inhibition of the H⁺, K⁺-ATPase (proton pump) enzymatic system of the secretory surface of gastric parietal cells. PPIs are advantageous substitutes for histamine H2 receptor antagonists (blocking of gastric acid secretion) or for antacids, which are not entirely efficient in the treatment of ulcers, associated or not associated with a Helicobacter pylori infection or other gastric conditions and which, in addition, lead to many side effects.

The PPIs to which the present invention more particularly relates are benzimidazole derivatives.

In the present disclosure, “benzimidazole derivative” denotes without distinction any substituted or unsubstituted benzimidazole PPI—with the exclusion of lansoprazole—per se, one or more salts of these benzimidazole PPIs, any enantiomer of these benzimidazole PPIs, one or more salts of enantiomer(s), any isomer of these benzimidazole PPIs, any benzimidazole derivative, any free base of a benzimidazole PPI, or any mixture of these active agents. By convention, the term “PPI” or “benzimidazole derivative” used in the singular in the present disclosure will denote without distinction one or more PPIs.

For example, the active ingredients to which the present invention relates are in particular the PPIs described on pages 7 to 11 of WO-A-97/25066, this passage being incorporated into the present disclosure by way of reference.

WO-A-2004/035020 gives a general formula of PPI of the benzimidazole class: pages 35-48, formula I′. This passage of WO-A-2004/035020 is integrated into the present disclosure by way of reference. By way of nonlimiting examples of PPI, mention may be made of the following products: esomeprazole, leminoprazole, omeprazole, pantoprazole, pariprazole, rabeprazole. Examples that may also be mentioned are timoprazole, picoprazole, tenatoprazole and ilaprazole.

PPIs are lipophilic weak bases which undergo rapid degradation under acidic conditions but which, on the other hand, are relatively stable at neutral or basic pH.

Ideally, a PPI-based oral medicament should have the following characteristics:

a) quickly provide relief to the patient by increasing the gastric pH after oral administration of the medicament;
b) accelerate the recovery of patients while maintaining this increase in gastric pH, for example above pH=4.0, for as long as possible after oral administration of the medicament, and in particular during the night;
c) improve the observance of the treatment and the comfort of the patient through the medicament being taken once daily.

For this, a PPI-based oral medicament should, firstly, sufficiently protect the PPI against the acidic conditions in the stomach, before its absorption downstream of the stomach, and, secondly, make it possible to obtain a plasma profile which is maintained above the effective therapeutic concentration, for as long as possible, in order to maximize the duration of action of the PPI, and therefore its therapeutic efficacy. This aim comes up against the limited residence time of the PPI in the blood compartment. For example, the plasma half-life of omeprazole is 0.5-1H.

In order to overcome this, the bioabsorption time of the PPI should therefore be prolonged by means of a judicious adjustment of its release in front of its bioabsorption window, in the upper parts of the gastrointestinal tract.

Up until now, it has commonly been accepted that PPIs, such as benzimidazole derivatives, that are unstable under acidic conditions (stomach) should be encapsulated in gastroresistant, i.e. enteric, coatings. However, these known enteric forms rapidly release the PPI when they enter the intestine and do not make it possible to ensure a satisfactory increase in gastric pH over 24 h or more, in particular during the night. In addition, these enteric forms do not make it possible to quickly provide relief to the patient.

Moreover, it is known practice to formulate the PPI with buffers in order to protect it against acids. However, while these formulations protect the PPI, they do not make it possible to maintain the increased gastric pH for a very long time.

A first category of PPI-based pharmaceutical preparations concerns the solid monolithic forms in which the active ingredient is coated with an enteric layer which protects it against degradation under the acidic pH conditions of the stomach.

Thus, for example, WO-A-97/25066 proposes a tablet containing microgranules of omeprazole (sprayed onto a neutral core of sugar), each coated successively with at least one separating layer (hydroxypropylmethyl-cellulose/talc/magnesium stearate), with at least one enteric layer (copolymer of methacrylic acid/mono- and diglycerides/triethyl citrate/polysorbate) and an external layer (hydroxypropylmethylcellulose/magnesium stearate). In order to produce the tablet, these microgranules are compressed after having mixed them with an antacid (aluminum hydroxide/magnesium carbonate) or with an alginate.

This type of enteric formulation has several deficiencies:

Since the dissolving of the enteric coating is pH-dependent and since the gastrointestinal pH exhibits a very great interindividual and intraindividual variability, this may result in a great variability, in vivo, of the pharmacokinetic profile.

This variability is further increased by the interindividual and intraindividual variability of the gastric residence time of monolithic oral forms.

Moreover, the enteric coatings can delay the absorption and therefore the beginning of the therapeutic activity of the PPI.

In addition, the tablet based on enteric microcapsules of PPI and of antacid according to WO-A-97/25066 neither deals with nor, a fortiori, solves the problem of the desired increase in the amount of time during which the gastric pH should exceed the value of pH=4.0.

Briefly, this document WO-A-97/25066 does not fully satisfy the desired characteristics a), b) and c) mentioned above.

A second category of PPI-based pharmaceutical preparations concerns the nonenteric forms in which the degradation of the PPI in an acidic medium is inhibited by the addition of another active compound that acts as a buffer.

Thus, PCT patent application WO-A-02/053097 proposes decreasing the interindividual and intraindividual variability by eliminating the enteric coating and combining the immediate-release PPI with a buffering agent that combines a bicarbonate salt of a metal of group IA and a carbonate salt of a metal of group IA.

This PCT application aims to propose a stable, nonenteric formulation of lansoprazole which allows a faster action than the enteric formulations. This formulation does not make it possible to increase the action time of lansoprazole.

A third category of formulations based on proton pump inhibitors concerns osmotic nonenteric systems.

PCT application WO-A-00/78293 describes oral pharmaceutical forms comprising microgranules each containing omeprazole mixed with an alkaline agent and one or more swelling agents (crosslinked polyvinyl-pyrrolidone), film-coated with a semipermeable membrane composed only of ethylcellulose and of talc. Under the effect of the swelling of the crosslinked polyvinylpyrrolidone, the ethylcellulose membrane ruptures and the omeprazole can be released. Application WO-A-00/78293 describes a release of 60% of the active ingredient in 2 hours, after a 2-hour exposure in an acidic medium, which simulates the residence time in the gastric environment. This PCT application does not therefore disclose any release which begins in the stomach, but rather a nonenteric release triggered by a time-dependent mechanism of swelling of the swelling agent.

A fourth category of PPI formulations concerns sustained-release preparations.

PCT application WO-A-2004/035020 proposes a formulation, in particular of PPI, in which the release of the active ingredient is controlled and which also contains a gelling polymer. The control release is obtained by coating the core containing the active ingredient with an enteric layer or with a layer of polymer controlling the kinetics of diffusion of the active ingredient to the outside, or else by dispersion of the active ingredient in an insoluble polymer matrix through which the active ingredient diffuses. The role of the gelling agent contained in the formulation according to WO-A-2004/035020 is to increase the residence time of the particles containing the active ingredient in the gastrointestinal tract and to thus make it possible to release it for a longer period of time in front of its absorption windows.

For example, the granule is coated with an enteric film composed of a methacrylic copolymer Eudragit®LD (enteric)/talc/TiO₂/polysorbate/macrogol. This enteric coating deposited onto PPI microparticles can itself be coated with a layer based on Eudragit®S (gelling polymer)/Eudragit®L (gelling polymer)/talc/triethyl citrate.

This invention teaches nothing with regard to the target value that this gastric pH must exceed and the means for keeping it high. More specifically, this document WO-A-2004/035020 does not fully satisfy the desired characteristics a), b) and c) mentioned above.

Patent U.S. Pat. No. 6,274,173 describes oral pharmaceutical compositions with delayed release of a specific benzimidazole derivative: pantoprazole, combined with an antimicrobial agent for the treatment of pathologies caused by Helicobacter. The pharmaceutical forms, tablets or granules, with delayed release of pantoprazole comprise a core based on pantoprazole, on sodium carbonate, on mannitol, on HPMC 2910-3, on HPMC 2910-15 and on calcium stearate, a sustained-released insoluble intermediate layer based on ethylcellulose, on lactose, on propylene glycol and on aqueous ammonia, and an enteric external coating based on Eudragit®L and on triethyl citrate.

This patent also discloses microcapsules consisting of a core based on particles of sugar coated with HPMC, with propylene glycol and with sodium hydroxide. This core is then coated with an active layer comprising pantoprazole, HPMC, propylene glycol and sodium hydroxide. An enteric coating based on Eudragit®L and on triethyl citrate is applied to the active layer in order to form microcapsules or “pellets” which are then packaged in gelatin capsules. A sustained-release layer can be interposed between the active layer and the enteric layer. The examples concern pantoprazole, but the claims of this patent exclude pantoprazole and are directed toward PPIs, in particular omeprazole and lansoprazole. Also claimed is a form with delayed and controlled release of PPI other than pantoprazole, combined with a form with immediate release of said PPI. The pharmaceutical forms disclosed by this patent are all forms with controlled and delayed release, comprising an enteric layer, and which do not allow release of the PPI in the stomach.

Application WO-A-99/32091 describes oral pharmaceutical forms, in particular tablets with sustained release of PPI, that are film-coated with an enteric membrane. WO-A-99/32091 discloses, inter alia, in vitro release rates such that 70% of omeprazole is released in 10 hours. The sustained release of the PPI is a matrix-based release, provided by dispersion of the active ingredient in a hydrophilic and/or hydrophobic polymer matrix. This polymer matrix can also contain an antacid which maintains the environment of the PPI at a pH at least equal to 7. The matricial core, containing the omeprazole and the antacid, is film-coated with an enteric film, which protects the active ingredient against the acidic gastric environment, and optionally with an intermediate layer based on a water-soluble polymer, such as hydroxypropylmethylcellulose, which separates the matricial core from the enteric layer. Said PCT application proposes pharmaceutical forms of PPI for which the sustained release begins only once it has left the stomach.

Application WO-A-2004/035090 discloses:
a compressed pharmaceutical formulation comprising:
   a PPI in the form of microgranules with modified release, in particular, of lansoprazole or of omeprazole (as described in PCT application WO-A-97/25066 mentioned above), coated with an enteric layer, with an intermediate separating layer between the enteric film and the PPI core (composed, for example, of hydroxypropylmethyl-cellulose/talc/magnesium stearate) and, optionally, with a water-soluble surrounding layer (hydroxypropylmethylcellulose/magnesium stearate), and
   an H2 receptor antagonist (cimetidine or famotidine), said antagonist being immediate-release;
a compressed pharmaceutical formulation comprising:
   a PPI in the form of microgranules with modified release, in particular, of lansoprazole or of omeprazole (such as described in patent U.S. Pat. No. 6,274,173 mentioned above), coated with a coating layer of insoluble polymer, based on ethylcellulose or on polyvinyl acetate, which slows down the release of the active ingredient, and with an enteric layer, and
   an H2 receptor antagonist (cimetidine or famotidine), said antagonist being immediate-release.

Application WO-A-2004/035090 teaches nothing regarding the performance levels of these formulations in the treatment of gastric ulcers and gastric pain and of disorders pertaining thereto, and in particular nothing regarding their abilities to satisfy the abovementioned characteristics a), b) and c).

Furthermore, these formulations do not allow release of the PPI in the stomach.

Patent EP-A-1 086 694 proposes microgranules of PPI that are film-coated with at least one coating layer composed of a mixture of soluble polymers (such as hydroxypropylcellulose or hydroxypropylmethylcellulose) and/or insoluble polymers (such as ethylcellulose and copolymers of ammonium methacrylate) and an enteric outer layer. This film-coating makes it possible to slow down the rate of release of the omeprazole, it being possible for said release to begin only once the formulation has left the stomach, due to the presence of the enteric layer.

European patent EP-B-0 709 087 discloses microcapsules whose core comprises an active ingredient such as a PPI used as an antiulcer agent (for example, omeprazole), in which the composition of the core-coating film comprises from 60% to 80% of ethylcellulose, from 5% to 10% of polyvinylpyrrolidone, from 5% to 10% of castor oil and from 2% to 8% of magnesium stearate. These microcapsules have a particle size of between 50 and 1000 microns and are designed in such a way as to be able to stay in the small intestine for a period of 5 and 24 hours, i.e. from 2 to 12 times longer than the natural transit time. This result is particularly advantageous. However, said European patent EP-B-0 709 087 teaches nothing regarding the performance levels of these formulations in the treatment of gastric ulcers and gastric pain and of disorders pertaining thereto, and in particular nothing regarding their abilities to satisfy the abovementioned characteristics a), b) and c).

In this context, it has to be noted that no oral medicament or pharmaceutical formulation exists that fully meets the abovementioned specifications, and in particular the characteristics a), b) and c).

The PPI-based medicinal solutions proposed to date are not therefore entirely satisfactory with regard to patients' expectations in terms of quick and long-lasting relief of gastric pain (burning) and of disorders pertaining thereto (bleeding).

### OBJECTIVES AND BRIEF DESCRIPTION OF THE INVENTION

Thus, one of the objectives of the invention is to provide an improved oral medicament for the treatment of gastric ulcers and gastric pain and of disorders pertaining thereto, more specifically an oral medicinal product with modified release of PPI.

Another objective of the invention is to provide an oral medicament with modified release of PPI which extends the bioabsorption time compared with a conventional enteric form, so as to maintain the gastric pH at an increased value for at least 24 h, and in particular overnight.

Another objective of the invention is to provide an oral medicament with modified release of PPI which quickly provides long-lasting relief to the patient for gastric pain (burning) or epigastric pain (reflux), and for disorders pertaining thereto (bleeding), by satisfying in particular the following characteristics:

a) quickly providing relief to the patient by increasing the gastric pH after oral administration of the medicament;
b) accelerating the recovery of patients while maintaining this increase in gastric pH for as long as possible after oral administration of the medicament, and in particular during the night;
c) improving the observance of the treatment and the comfort of the patient through the medicament being taken once daily.

Another objective of the invention is to provide a nonenteric oral medicament with modified release of PPI, which results, in the patient, in an increase in the pH that is sufficient and over a longer period of time than that obtained with the known PPI-based formulations.

Another objective of the invention is to provide a nonenteric oral medicament with modified release of PPI, of the matricial or reservoir system type and which results, in the patient, in an increase in the pH that is sufficient and over a longer period of time than that obtained with the known PPI-based formulations.

Another objective of the invention is to provide a multimicrocapsular oral medicament containing a plurality of nonenteric microcapsules of PPI with modified release, said formulation having an in vitro release profile independent of the dose administered.

Another objective of the invention is to provide a multimicrocapsular oral medicament containing a plurality of nonenteric microcapsules of PPI with modified release, it being possible for these microcapsules to be administered as a mixture with excipients and/or a buffering agent.

These objectives, among others, are attained by means of the invention, which relates to a PPI-based oral medicament allowing modified release of this PPI, characterized in that it is designed such that, after its ingestion as a once-daily dose, it makes it possible to maintain, from the first day of treatment onward, an average gastric pH, between 0 and 24 h, that is greater than or equal to the average gastric pH, between 0 and 24 h, obtained with a reference* immediate-release enteric oral medicament administered under the same conditions.

According to another pharmacodynamic definition of the invention, the medicament according to the invention is designed such that, after its ingestion, the release of the PPI begins in the stomach and that, when it is administered as a once-daily dose in the morning, it makes it possible to maintain, from the fifth day of treatment onward, the average gastric pH, between 16 hours and 20 hours after the dose has been taken, at greater, preferably greater by at least 0.5 pH unit, and even better still greater by at least 1 pH unit, than the average gastric pH, between 16 hours and 20 hours after the dose has been taken, obtained with a reference* immediate-release enteric oral medicament administered under the same conditions.

This medicament, which may or may not satisfy the definition given in the previous paragraphs, can also be designed such that, after its ingestion as a once-daily dose, it makes it possible to maintain, from the first day of treatment onward, a gastric pH that is greater than or equal to the gastric pH obtained with a reference* immediate-release enteric oral medicament administered under the same conditions, for at least 16 h, preferably at least 20 h, and even more preferably at least 22 h, within the dosage period (i.e. 24 hours for a medicament administered as a once-daily dose). This period during which the gastric pH corresponds to the previous condition may be continuous or discontinuous. The period is considered to be cumulative during the dosage period.

This medicament, which may or may not satisfy the definition given in the previous paragraphs, can also be designed such that, after its ingestion as a once-daily dose, it makes it possible to maintain, from the fifth day of treatment onward, a gastric pH that is greater than or equal to the gastric pH obtained with a reference* immediate-release enteric oral medicament administered under the same conditions, for at least 13 h, preferably at least 16 h, and even more preferably at least 20 h, within the dosage period (i.e. 24 hours for a medicament administered as a once-daily dose). This period during which the gastric pH corresponds to the previous condition may be continuous or discontinuous. The period is considered to be cumulative during the dosage period.

The expression “reference* immediate-release enteric oral medicament” denotes, in the present disclosure, an enteric medicament, which releases the same PPI as the medicament according to the invention, releasing at pH 6.8 and under SINK conditions in an in vitro dissolution test, most of the amount of the PPI that it contains in a relatively brief period of time; for example, at least 70% is released in 45 minutes, preferably in thirty minutes.

### DETAILED DESCRIPTION OF THE INVENTION

The comparison between the medicament according to the invention and the reference* immediate-release enteric oral medicament, as regards the increase in gastric pH, can be carried out by means of a reference clinical T1 test. The experimental conditions of the T1 test can, for example, be the following: once-daily administration for five days to 30 normal human volunteers in the course of a crosstrial study. The gastric pH is measured by a Digitrapper® pH100 probe, every 4 seconds over 24 hours post-administration at days No. 1 and No. 5. The nonphysiological aberrant pH points are considered to be not measured. The average gastric pH is then calculated from the values collected.

The period during which the medicament according to the present invention makes it possible to maintain a gastric pH that is greater than or equal to the gastric pH obtained with a reference* immediate-release enteric oral medicament, administered under the same conditions, is estimated in the following way:

The gastric pH profiles measured in the T1 test for the medicament according to the invention and for the reference formulation are first of all polished by replacing the value of the pH at each instant t by its mean taken over the time period t−15 minutes; t+15 minutes. The time during which the formulation according to the present invention gives a pH greater than the pH given by the reference formulation is then measured on these polished profiles.

This clinical test defines the invention by the pharmacodynamic properties obtained specifically under the conditions of the T1 test. Nevertheless, the invention is not limited to an implementation under the conditions of this T1 test.

The modified-release medicaments according to the invention are, for example, systems of reservoir type or of matrix type.

For the purpose of the present invention, the term “system of reservoir type” is intended to mean a system in which the volume of material containing the PPI is completely coated with at least one film which controls the rate of release of the PPI by diffusion of the PPI through the film, and which does not contain any PPI. This release takes place following the system coming into contact with the fluids of the gastrointestinal tract. The material containing the PPI is, for example, the PPI itself or a mixture of pharmaceutical excipients and of PPI. The reservoir system comprises, for example, a plurality of individually coated microcapsules or a monolithic system such as one (or more) coated tablet(s) or alternatively a tablet or any other pharmaceutical form containing a plurality of coated microcapsules.

For the purpose of the present invention, the term “matrix system” is intended to mean a system in which the PPI is dispersed in a polymeric phase known as a matrix, which controls the rate of release of the PPI. This matrix may be nonerodible or erodible.

The matrix is composed of pharmaceutical excipients known to those skilled in the art. The matrix system comprises, for example, a plurality of matrix microgranules (matrix elements) containing the PPI, which are uncoated or not entirely coated, with at least one film. The matrix system can also, for example, be a monolithic system (matrix element) such as one (or more) tablet(s) not entirely coated with at least one continuous film, containing no reservoir system. The matrix system can thus be, for example, a tablet comprising PPI granules with immediate and sustained release, dispersed in a polymeric matrix.

Preferably, the PPI-based oral medicament according to the invention is a reservoir-type system, comprising a plurality of microcapsules (reservoir elements) with modified release of PPI, these microcapsules individually comprising at least one microparticle containing PPI and coated with at least one coating which allows modified release of the PPI.

In the present disclosure, the expression “reference* immediate-release enteric oral medicament” denotes a medicament which releases the same PPI as the medicament according to the invention, and in which the microcapsules, or the pharmaceutical form, comprise an enteric coating which releases, at pH 6.8 and under SINK conditions in an in vitro dissolution test, most of the amount of PPI contained in the microcapsules, in a relatively brief period of time; for example, at least 70% of the PPI is released in 45 minutes, preferably in thirty minutes.

All the dissolution profiles to which reference is made in the present disclosure are carried out according to the indications of the European Pharmacopoeia 4th edition entitled: “Dissolution test for solid oral forms”: type II dissolutest carried out under SINK conditions at 37° C., at a test dose of 10, 40 or 80 mg of PPI, and unless otherwise mentioned, with stirring at 100 rpm.

In the disclosure of the invention, the term “microcapsules” denotes microparticles comprising PPI and film-coated with at least one coating which allows the modified release of PPI. The non-film-coated PPI microparticles can, for example, be neutral cores coated with at least one layer containing the PPI, or microparticles of pure PPI or else granules formed by a matrix of carrier excipients including the PPI.

Advantageously, the covering(s) for film-coating (or coating) has (have) a mechanical strength sufficient to prevent it (them) from tearing and/or from breaking up in the organism, until the end of the release of the active ingredient.

These reservoir microcapsules can be compared to carriers which allow the transport and the modified release of PPI and, optionally, of one or more other active ingredients in the stomach and in the small intestine.

According to a notable embodiment of the invention, this medicament is designed such that, after its ingestion, the release of the PPI begins in the stomach and that, when it is administered as a once-daily dose, it makes it possible to maintain, from the fifth day of treatment onward, or even from the first day of treatment onward, the gastric pH at a value greater than or equal to 4.0, for a period of time D greater than or equal to the period of time D* during which the pH is maintained at a value greater than or equal to 4.0 with a reference* immediate-release enteric oral medicament administered under the same conditions, D preferably being greater than or equal to D* by at least 5% (% relative to D), more preferably by at least 10%, and even more preferably by at least 20%.

According to another notable embodiment of the invention, this medicament is characterized in that it is nonenteric.

According to a preferred form of this 2nd embodiment, the medicament is characterized in that it comprises microcapsules with modified release of PPI and in that the coating of the PPI microcapsules is nonenteric.

For the purpose of the invention, the expression “enteric medicament or coating” is intended to mean a gastroresistant medicament or coating which does not release under the gastric conditions and which releases in the small intestine. Many published patent applications, including in particular those mentioned above, describe enteric medicaments or coatings. The US Pharmacopeia: “USP 28 NF 23 EDITION 2005” also gives a definition of an enteric medicament or coating.

In particular, a nonenteric medicament or coating, for the purpose of the invention, can for example be a medicament or coating which does not contain any enteric polymer in significant amount, i.e. in an amount sufficient to be effective in particular from a physicochemical point of view. The term “enteric” polymer denotes a polymer which is insoluble at a pH less than or equal to 5, 5.5, 6 or 7, respectively, depending on the nature of the polymer, and which is soluble above this pH.

The administration of the medicament, preferably multimicrocapsular medicament, according to the invention to patients or individuals results in an increase in their gastric pH, for at least 24 h, and in particular overnight. This increase in the gastric pH can be monitored by measuring the gastric pH in situ, by means of a gastric probe.

The medicament according to the invention can also be characterized by the plasma concentration profile, obtained in a reference T2 clinical test according to which the medicament is administered orally to a sample of N human individuals, preferably N≧20 or 30 individuals. The individual plasma concentration profile of each of the patients is then measured, and from this are drawn the individual pharmacokinetic parameters such as the time Tmax after which the plasma concentration reaches its maximum and the value of this maximum concentration Cmax. Based on these individual parameters, the person skilled in the art conventionally calculate the mean values of these parameters and their standard deviations. Further details on the discussion of these parameters will be found in the publication: Pharmacokinetics and Pharmacodynamic Data Analysis, 3rd ed., J. Gabrelsson et al., Kristianstadsi Bocktryckeri A B, Sweden, 2000.

The experimental conditions of the T2 clinical test can, for example, be the following: administration of the form (gelatin capsule, tablet, sachet or suspension) once a day, before breakfast, to 20 normal human volunteers in the course of a crosstrial study. The plasma concentrations of PPI are measured at the times: 0-0.25-0.5-0.75-1-1.5-2-3-4-6-8-10-12-16-18-20-24-36-48 hours post-administration. This T2 clinical test defines the invention by the pharmacokinetic properties obtained specifically under the conditions of the test. Nevertheless, the invention is not limited to an implementation under the conditions of this T2 clinical test.

Thus, with reference to this T2 test, the medicament according to the invention is characterized in that it is designed such that, when it is administered as a once-daily dose, it makes it possible to obtain, after the dose has been taken, a plasma profile defined as follows:

Cmax/C12h≦Cmax*/C12h*

preferably

1.5×Cmax/C12h≦Cmax*/C12h*

and even more preferably

2.0×Cmax/C12≦Cmax*/C12h*

with
C12h representing the mean plasma concentration of PPI 12 h after the dose has been taken,
C12h* representing the mean plasma concentration of PPI, obtained under the same conditions as C12h, with a reference* immediate-release enteric oral medicament containing the same dose of the same PPI,
Cmax representing the mean maximum plasma concentration of PPI after the dose has been taken,
Cmax* representing the mean maximum plasma concentration of the same PPI, obtained under the same conditions as Cmax, with a reference* immediate-release enteric oral medicament containing the same dose of the same PPI.

In the present disclosure, the term “modified release” is intended to denote a release of PPI by an oral medicament in which 70% of the PPI is released, in an in vitro dissolution test at pH 6.8, in a time greater than or equal to 45 minutes.

A modified-release medicament may, for example, comprise an immediate-release phase and a slow-release phase.

Modified-release medicaments are well known in this field; see, for example, Remington: The science and practice of pharmacy, 19th edition, Mack Publishing Co. Pennsylvania, USA.

The modified release can in particular be a sustained and/or delayed release.

The multimicrocapsular medicament according to the invention can also be characterized in that the PPI microcapsules have an in vitro release profile in potassium dihydrogen phosphate/sodium hydroxide (0.05M) buffer medium, at pH 6.8, such that:
70% of the PPI is released in a time of between 1 and 10 hours, preferably between 2 and 8 hours, and even more preferably between 2 and 6 hours, and
40% of the PPI is released in a time of between 0.5 and 5 hours, preferably between 1 and 4 hours, and even more preferably between 1 and 3 hours.

According to a specific definition in the case where the microcapsules contain omeprazole as PPI, the omeprazole microcapsules have an in vitro release profile in potassium dihydrogen phosphate/sodium hydroxide (0.05M) buffer medium, at pH 6.8, such that:
70% of the omeprazole is released in a time of between 2 h and 8 h, preferably between 2 h and 5 h,
40% of the omeprazole is released in a time of between 1 h and 4 h, preferably between 1 h and 3 h,
at least 70% of the omeprazole, preferably at least 90% of the omeprazole, is released in 10 h.

According to another definition of the medicament according to the invention, the PPI microcapsules have an in vitro release profile in potassium dihydrogen phosphate/sodium hydroxide (0.05M) buffer medium, at pH 6.8, such that, for any value of the time t of between 2 h and t(70%), preferably for any value of the time t of between 1 h and t(70%), the % of dissolved (released) PPI is greater than or equal to 35t/t(70%); i.e. the profile according to the invention releasing 70% of the PPI at the time referred to as t(70%) remains above a linear profile releasing half as much PPI (i.e. 35%) in the same time t(70%), this being the case at any instant between 2 h and t(70%), preferably at any instant between 1 h and t(70%).

Advantageously, the medicament comprises at least one external buffering agent.

For the purpose of the invention, the term “external buffering agent” will denote, in the plural as well as in the singular, a single compound or a mixture of compounds. This external buffering agent, formulated and/or administered with the matrix or reservoir elements (e.g. microcapsules) with modified release of PPI, has the effect of preventing the acidic degradation of the PPI and of preserving its bioavailability.

For the purpose of the invention, the description “external” indicates that the buffering agent is outside the matrix or reservoir elements (e.g. microcapsules). A buffering agent contained in the matrix or reservoir elements (e.g. microcapsules) cannot therefore be described as an external buffer for the purpose of the invention. The external buffering agent is present in the medicament according to the invention in the form of one or more individualized structures separate from the matrix or reservoir elements (e.g. microcapsules).

Preferably, the medicament according to the invention consists of one or more same pharmaceutical units (e.g. tablet, gelatin capsule or sachet) each containing, firstly, the matrix or reservoir elements (e.g. microcapsules) and, secondly, the external buffering agent.

However, according to another variant, it is possible to envision that the medicament according to the invention consists, firstly, of one or more pharmaceutical units (e.g. tablet, gelatin capsule or sachet) each containing the matrix or reservoir elements (e.g. microcapsules) and, secondly, of one or more pharmaceutical units (e.g. tablet, gelatin capsule or sachet) each containing the external buffering agent.

According to another variant, the medicament according to the invention comprises:
one or more same pharmaceutical units (e.g. tablet, gelatin capsule or sachet) each containing, firstly, the matrix or reservoir elements (e.g. microcapsules) and, secondly, the external buffering agent;
and one or more pharmaceutical units (e.g. tablet, gelatin capsule or sachet) each containing the matrix or reservoir elements (e.g. microcapsules and/or one or more pharmaceutical units (e.g. tablet, gelatin capsule or sachet) each containing the external buffering agent.

According to one variant, the medicament comprises at least one matrix element without external buffer.

The medicament according to the invention can also be in the form of a multidose oral suspension, reconstituted from powder and water before administration.

For the needs of the invention, the external buffering agent advantageously comprises at least one weakly or strongly basic, pharmaceutically acceptable compound.

For example, the external buffering agent can be chosen from the following list, without however being limited thereto:

amino acids and their salts, sodium salts, potassium salts, calcium salts, magnesium salts, aluminum salts, these salts preferably being selected from the following salts: hydroxides, oxides, lactates, gluconates, carbonates, sesquicarbonates, bicarbonates, silicates, phosphates, glycerophosphates, pyrophosphates, polyphosphates or chlorides. The buffering agent can naturally be a mixture of all or some of these compounds.

The external buffering agent or a part of the external buffering agent preferably has a high buffering capacity, for example greater than or equal to 10 mEq/g (milliequivalent/g), preferably greater than or equal to 20 mEq/g, and preferentially greater than or equal to 40 mEq/g. Such buffering agents make it possible to prepare pharmaceutical units of reasonable size, which are not unacceptable in terms of swallowing thereby improving the observance and therefore the success of the treatment.

The external buffering agent is preferably calcium carbonate, optionally combined with magnesium oxide or magnesium hydroxide.

According to a notable feature of the invention, the medicament contains between 0 and 100 mEq, preferably between 2 and 40 mEq, of external buffering agent.

According to a first preferred embodiment, the external buffering agent comprises calcium carbonate.

In accordance with an advantageous variant of this first preferred embodiment, the calcium carbonate is present in a proportion of 2 to 15 mEq, preferably 5 to 10 mEq.

According to a second preferred embodiment, the external buffering agent comprises magnesium oxide.

Advantageously, the external buffering agent comprises between 5 and 35 mEq, preferably between 5 and 25 mEq of magnesium oxide.

According to a third embodiment, the external buffering agent comprises from 3 to 7 mEq of calcium carbonate and an amount of magnesium oxide such that the magnesium oxide/calcium carbonate ratio, in milliequivalents, is between 1.5 and 5.

According to a nonlimiting example, the external buffering agent comprises approximately 5 mEq of calcium carbonate and approximately 12.5 mEq of magnesium oxide.

According to a fourth embodiment, the external buffering agent selected comprises magnesium hydroxide.

Advantageously, the external buffering agent selected comprises between 5 mEq and 30 mEq, preferably between 5 mEq and 20 mEq, of magnesium hydroxide.

According to a fifth embodiment, the external buffering agent selected comprises from 3 to 7 mEq of calcium carbonate and an amount of magnesium hydroxide such that the magnesium hydroxide/calcium carbonate ratio, in milliequivalents, is between 1.5 and 5.

According to a nonlimiting example, the external buffering agent selected comprises approximately 5 mEq of calcium carbonate and approximately 8.5 mEq of magnesium hydroxide.

In practice, the buffering agent is, for example, immediate-release.

Advantageously, the matrix or reservoir elements (e.g. microcapsules) of PPI can contain at least one internal buffering agent. Unlike the external buffering agent, the internal buffering agent is an integral part of the matrix or reservoir elements (e.g. microcapsules).

Said internal buffering agent is selected from weakly or strongly basic, pharmaceutically acceptable compounds, and chosen, for example, from the list of buffering agents mentioned hereinafter:

amino acids and their salts, sodium salts, potassium salts, calcium salts, magnesium salts, aluminum salts, these salts preferably being selected from the following salts: hydroxides, oxides, lactates, gluconates, carbonates, sesquicarbonates, bicarbonates, silicates, phosphates, glycerophosphates, pyro-phosphates, polyphosphates or chlorides. The buffering agent can naturally be a mixture of all or some of these compounds.

This internal buffering agent preferably comprises magnesium hydroxide.

This internal buffering agent in direct contact with the PPI has the effect of preventing any acidic degradation of the PPI, that may occur inside the matrix or reservoir elements (e.g. microcapsules).

Preferably, the coating of the reservoir elements (e.g. microcapsules) comprises at least one layer which controls the modified release of PPI, the composition of which is the following:

A. at least one film-forming (co)polymer (A) which is insoluble in the fluids of the gastrointestinal tract;
B. optionally at least one hydrophilic film-forming (co)polymer (B) which
is insoluble in the fluids of the gastrointestinal tract,
bears groups which are ionized in the fluids of the gastrointestinal tract;
   C. at least one (co)polymer (C) which is soluble in the fluids of the gastrointestinal tract;
   D. at least one plasticizer (D);
   E. optionally at least one surfactant and/or lubricant (E).

According to a preferred embodiment of the invention:

(A) is selected from the group of following products:
water-insoluble derivatives of cellulose, preferably ethylcellulose and/or cellulose acetate,
polyvinyl acetates,
and mixtures thereof;
   (B) is chosen from water-insoluble charged acrylic polymers, preferably from (co)polymers of an ester of acrylic and/or methacrylic acid bearing at least one quaternary ammonium group; (B) even more preferably comprising at least one copolymer of alkyl (meth)acrylate and of trimethylammonioethyl meth-acrylate chloride, and more specifically the products sold under the trade marks Eudragit® RS and/or Eudragit® RL [copolymers of an acrylic acid ester (ethyl acrylate) and of a (meth)acrylic acid ester (methyl (meth)acrylate) and of trimethylammonioethyl methacrylate chloride], e.g. the powders Eudragit® RL PO and/or Eudragit® RS PO and/or the granules Eudragit® RL 100 and/or Eudragit® RS 100 and/or the suspensions and/or solutions of these Eudragits® RL and Eudragits® RS, i.e., respectively, Eudragit® RL 30D and/or Eudragit® RS 30D and/or Eudragit® RL 12.5 and/or Eudragit® RS 12.5;
   (C) is chosen from:
nitrogenous (co)polymers, preferably from the group comprising polyacrylamides, poly-N-vinylamides, polyvinylpyrrolidones (PVPs) and poly-N-vinyllactams;
water-soluble derivatives of cellulose,
polyvinyl alcohols (PVAs),
polyoxyethylenes (POEs),
polyethylene glycols (PEGs),
hydrocolloids, such as xanthan gums, guar gums, pectins, carob gum, carrageenans, gelatin, agar agar, modified or unmodified starches, dextrins or alginates,
and mixtures thereof,
   polyvinylpyrrolidone, polyoxyethylenes, polyethylene glycols and hydroxypropylcellulose being particularly preferred;
   (D) is chosen from the group comprising:
cetyl alcohol esters,
glycerol and esters thereof, preferably from the following subgroup: acetyl glycerides, glyceryl monostearate, glyceryl triacetate (triacetin), glyceryl tributyrate,
phthalates, preferably from the following subgroup: dibutyl phthalate, diethyl phthalate, dimethyl phthalate, dioctyl phthalate,
citrates, preferably from the following subgroup: acetyltributyl citrate, acetyltriethyl citrate, tributyl citrate, triethyl citrate,
sebacates, preferably from the following subgroup: diethyl sebacate, dibutyl sebacate,
adipates,
azelates,
benzoates,
plant oils,
fumarates, and preferably diethyl fumarate,
malates, preferably diethyl malate,
oxalates, preferably diethyl oxalate,
succinates, preferably dibutyl succinate,
butyrates,
salicylic acid,
malonates, preferably diethyl malonate,
castor oil (the latter being particularly preferred),
and mixtures thereof;
   (E) is chosen from the group comprising:
anionic surfactants, preferably from the subgroup of alkali metal salts or alkaline earth metal salts of fatty acids, stearic acid and/or oleic acid being preferred,
and/or nonionic surfactants, preferably from the following subgroup:
   polyoxyethylenated oils, preferably polyoxy-ethylenated hydrogenated castor oil,
   polyoxyethylene/polyoxypropylene copolymers,
   polyoxyethylenated sorbitan esters,
   polyoxyethylenated castor oil derivatives,
   stearates, preferably calcium stearate, magnesium stearate, aluminum stearate or zinc stearate,
   stearyl fumarates, preferably sodium stearyl fumarate,
   glyceryl behenates,
   and mixtures thereof.

According to a particularly advantageous embodiment, the composition of the modified-release layer is the following:

A. the film-forming polymer(s) (A) is (are) present in a proportion of 10% to 90%, preferably 20% to 40% by weight on a dry basis, relative to the total mass of the coating composition;
B. the water-insoluble hydrophilic film-forming polymer(s) (B) is (are) present in a proportion of 10% to 90%, preferably 20% to 40% by weight on a dry basis, relative to the total mass of the coating composition;
C. the polymer(s) (C) which is (are) soluble in the fluids of the gastrointestinal tract is (are) present in a proportion of 2% to 25%, preferably 5% to 15% by weight on a dry basis, relative to the total mass of the coating composition;
D. the plasticizer(s) (D) is (are) present in a proportion of 2% to 20%, preferably 4% to 15% by weight on a dry basis, relative to the total mass of the coating composition;
E. the optional surfactant(s) and/or lubricant(s) (E) is (are) present in a proportion of 2% to 20%, preferably 4% to 15% by weight on a dry basis, relative to the total mass of the coating composition.

That said, in the case of reservoir microcapsules containing omeprazole as PPI, but without however being limited to this specific PPI, the modified-release layer can preferably have the following composition by mass:

A. the film-forming polymer(s) (A) is (are) present in a proportion of 40% to 55%, preferably 45% to 55% by weight on a dry basis, relative to the total mass of the coating composition;
C. the soluble polymer(s) (C) is (are) present in a proportion of 15% to 30%, preferably 20% to 30% by weight on a dry basis, relative to the total mass of the coating composition;
D. at least one plasticizer (D) is (are) present in a proportion of 3% to 10%, preferably 3% to 7% by weight on a dry basis, relative to the total mass of the coating composition;
E. the optional surfactant(s) and/or lubricant(s) (E) is (are) present in a proportion of 10% to 30%, preferably 15% to 25% by weight on a dry basis, relative to the total mass of the coating composition.

Preferably, in one embodiment of the invention in which the PPI envisioned is omeprazole, but without however being limited to this specific PPI, the coating of the reservoir elements (e.g. microcapsules) comprises at least one layer which controls the modified release of the omeprazole. The composition of this layer is the following:

(A) is selected from the group of following products: water-insoluble derivatives of cellulose, preferably ethylcellulose and/or cellulose acetate;
(C) is chosen from polyvinylpyrrolidones (PVPs) and water-soluble derivatives of cellulose, such as hydroxypropylcellulose; PVPs being preferred;
(D) is castor oil;
(E) is chosen from: polyoxyethylene/polyoxypropylene copolymers, preferably polyoxyethylene/polyoxypropylene block terpolymers.

For further details, in particular qualitative and quantitative details, on at least some of the constituents of this coating composition, reference will, for example, be made to European patent EP-B-0 709 087 or to PCT applications WO-A-2004/010983 and WO-A-2004/010984, the content of which is integrated into the present exposure by way of reference.

The monolayer or multilayer coating can comprise various other additional adjuvants conventionally used in the coating field. They may, for example, be pigments, dyes, fillers, antifoams, etc.

According to a specific embodiment of the invention, the coating of the microcapsules controlling the modified release of PPI consists of a single layer or a single film coating. This simplifies their preparation and limits the degree of coating.

Moreover, the medicament according to the invention has the particularity that the coating of each “reservoir” element (e.g. microcapsule) is nonenteric and does not disintegrate irrespective of the pH, and in particular at pH greater than or equal to 5.0.

The matrix of the matrix elements can comprise all the pharmaceutically acceptable excipients, and in particular those defined above for the composition of the reservoir elements.

Advantageously, the principal functional materials used for preparing these matrix systems correspond to the following groups:
hydrophilic polymers that are soluble in the fluids of the gastrointestinal tract, such as povidone, water-soluble derivatives of cellulose, xanthan gum, polyethylene glycols, polyvinyl alcohols, etc.,
hydrophobic polymers that are insoluble in the fluids of the gastrointestinal tract, such as water-insoluble derivatives of cellulose, water-insoluble (meth)acrylic polymers, polyvinyl acetates, etc.

Other excipients can also be integrated into the formulation of the matrix compounds, for instance disintegrating agents, lubricants, waxy compounds, dyes, plasticizers, fillers, pH modifiers, pH-sensitive compounds, surfactants, flavorings, etc.

Advantageously, the diameter of the microcapsules is less than or equal to 1000 μm, preferably between 5 and 800 μm, and even more preferably between 100 and 600 μm. In the specific case of microcapsules in which the PPI is omeprazole, the diameter of the microcapsules can be between 100 and 500 μm, preferably between 100 and 400 μm, and more preferably between 100 and 300 μm.

The diameters of the microparticles and of the microcapsules to which the present disclosure relates are, unless otherwise indicated, volume-average diameters.

According to the invention, practical embodiments in which the proportion of PPI in the microcapsules (expressed as % by weight on a dry basis relative to the total mass of the microcapsules) is between 5 and 95, preferably between 10 and 85, and even more preferably between 20 and 70, are preferred.

As regards the monolayer or multilayer coating (or film-coating) which controls the modified release of the PPI, it represents, for example, at most 40%, preferably at most 15% by weight of the microcapsules. According to a specific embodiment in which the microcapsules contain omeprazole as PPI, the modified-release layer represents from 2% to 25% by weight, preferably from 5% to 20% by weight, and more preferably from 5% to 15% by weight, relative to the total weight of the omeprazole microcapsules.

Such limited degrees of coating make it possible to prepare pharmaceutical units each containing a high dose of PPI, without exceeding a size that is completely unacceptable with regard to swallowing. The observance and therefore the success of the treatment can only be improved by this.

As regards the structure of the microcapsules used in the preferred multimicrocapsular embodiment of the medicament according to the invention, two preferred embodiments of the structure of the microcapsules are described in detail hereinafter, in a nonlimiting manner.

According to a first embodiment, at least a part of the microcapsules with modified release of PPI each comprise:
a microparticle of PPI, coated with
at least one coating which allows the modified release of PPI.

Preferably, the microparticle of PPI consists of crude (pure) PPI or a matrix granule of PPI with one or more other pharmaceutically acceptable ingredients.

According to a second embodiment, at least a part of the microcapsules with modified release of PPI each comprises:
a neutral core,
at least one active layer comprising the PPI and coating the neutral core, and
at least one coating which controls the modified release of PPI.

According to a first possibility, the neutral core contains sucrose and/or dextrose and/or lactose.

According to a second possibility, the neutral core is a cellulose microsphere.

Advantageously, the neutral core has an average diameter of less than or equal to 800 μm, preferably between 20 and 500 μm.

The active layer can optionally comprise, in addition to the PPI, at least one internal buffering agent and/or at least one active ingredient other than the PPI and/or one or more pharmaceutically acceptable excipients.

The optional internal buffering agent is, for example, included within the PPI microparticles (coated neutral core or granule), from which the microcapsules are prepared, in direct contact with the PPI. The internal buffering agent can, for example, be:
intimately mixed with the PPI in the active layer containing the PPI and covering a neutral core,
or included with the PPI in a matrix of excipients forming a granule,
or else film-coated onto microparticles of pure PPI,
or else incorporated, alone or as a mixture, into the core.

Advantageously, the techniques for depositing the coating which allows the modified release of PPI or for depositing the PPI-based active layer are techniques known to those skilled in the art, for example the fluidized air bed spray coating technique, wet granulation, compacting, extrusion-spheronization, etc.

The PPI microcapsules are all the more advantageous when they are also completely tolerated by the organism, in particular at the gastric level and, moreover, when they can be readily and economically obtained.

The medicament according to the invention can comprise, in addition to a PPI, one or more active ingredients. This (or these) other active ingredient(s) can be included in matrix elements or reservoir elements (e.g. microcapsules) with modified release of active ingredient(s), which may or may not comprise a PPI.

According to an advantageous variant of the invention, the PPI-based oral medicament also comprises at least one H2 receptor antagonist, preferably selected from the group comprising the following active ingredients: cimetidine, ranitidine, nizatidine, famotidine, pharmaceutically acceptable salts thereof, isomers thereof and salts of an isomer thereof, and also any mixture of these various active ingredients.

The medicament according to the invention can comprise microunits consisting of microcapsules with modified release of PPI and/or microunits of PPI, other than microcapsules, such as matrix (micro)granules. They could, for example, be microparticles with immediate release of PPI and/or of one or more other active ingredient(s). These immediate-release microparticles can be of the same type as those that can be used in the preparation of the microcapsules defined above.

In addition, the collection of microunits (microparticles and/or microcapsules) constituting the medicament according to the invention can be formed by various populations of microunits, these populations differing from one another at least by virtue of the nature of the active ingredient(s) other than the PPI that is (are) contained in these microunits and/or by virtue of the amount of PPI or of other optional active ingredient(s) that they contain and/or by virtue of the composition of the coating and/or by virtue of the fact that they are modified-release or immediate-release.

The microcapsules described above can be used for the manufacture of novel PPI-based pharmaceutical preparations or medicaments having optimized therapeutic performance levels, in particular with respect to gastric conditions, and which are preferably in various pharmaceutical forms: disintegratable or dispersible tablets, gelatin capsules, matrix tablets or granules, sachets or multidose suspensions to be reconstituted without the release profile of the PPI microcapsules being modified.

Advantageously, the medicament containing the microcapsules with modified release of PPI also comprises conventional pharmaceutically acceptable excipients known to those skilled in the art, that can be used, for example, for providing the microcapsules in tablet form. For example, these excipients can in particular be:
compression agents such as microcrystalline cellulose or mannitol,
dyes,
disintegrating agents such as crospovidone or crosslinked polyvinylpyrrolidone or crosslinked povidone; sodium croscarmellose or crosslinked sodium carboxymethylcellulose; sodium starch glycolate; pregelatinized cornstarch,
flow agents such as talc,
lubricants such as, for example, glyceryl behenate,
flavorings,
preserving agents,
and mixtures thereof.

When the medicament is in tablet form, said tablet can be coated according to the techniques and formulae known to those skilled in the art in order to improve its presentation: color, appearance, taste masking, etc.

Preferably, when the medicament is in tablet form, it comprises a plurality of microcapsules with modified release of PPI as described above. In a preferred variant, the tablet has an in vitro release profile in the potassium dihydrogen phosphate/sodium hydroxide (0.05M) buffer medium, at pH 6.8, similar to that of the microcapsules of said tablet, according to the similarity factor f2. For the record, the similarity test is defined as follows: the similarity between two dissolution profiles is evaluated by means of the similarity factor f2 as defined in the document “Quality of modified release products” from the European agency for the evaluation of medicinal products, document referenced CPMP/QWP/604/96 (Annex 3). An f2 value of between 50 and 100 indicates that the two dissolution profiles are similar.

In particular, the tablet contains, in addition to a plurality of microcapsules with modified release of PPI,
from 5 to 25 mEq of external buffering agent, preferably from 10 to 20 mEq of external buffering agent,
compression excipients in an amount such that the total mass of the tablet does not exceed 1000 mg, preferably 800 mg, and more preferably 600 mg.

In a variant of the medicament in tablet form, the external buffering agent is chosen from calcium carbonate, magnesium oxide and mixtures thereof.

Preferably, the tablet has a hardness greater, in an increasing order of preference, than 80N, 100N, 120N. Preferably, the hardness is less than 300N, better still less than 200N. According to a variant of the invention, the hardness of the tablet is between 100N and 150N.

As regards the dose, the medicament according to the invention can advantageously exist in the form of a once-daily oral dose comprising from 1 mg to 500 mg of PPI, without this being limiting.

The novel PPI-based medicaments according to the invention are original in terms of their structure, their presentation and their composition and can be administered per os, in particular in once-daily doses.

It should be noted that it may be advantageous to mix, in the same gelatin capsule, the same tablet or the same powder for oral suspension, at least two types of microcapsules with different PPI-release kinetics, but which are included in the characteristic context of the invention.

It may also be recalled that it is possible to mix the microcapsules according to the invention with a certain amount of PPI immediately available in the organism (immediate release).

Without wishing to be limiting, it should nevertheless be emphasized that the medicament according to the invention is particularly advantageous in that it can be:
in the form of a once-daily oral dose comprising from 100 to 500 000 microunits, some of which contain the PPI;
in the form of a once-daily oral dose comprising from 100 to 500 000 microcapsules with modified release of the PPI and, optionally, at least one other active ingredient.

Moreover, the invention is directed toward the use of the microparticles as defined above, for the preparation of microparticulate pharmaceutical oral galenic forms, preferably in the form of tablets, powders for oral suspension or gelatin capsules.

Finally, the invention is also directed toward a therapeutic treatment method, characterized in that it consists essentially of ingestion, according to a given dosage, of a medicament as defined above and comprising the microcapsules themselves also defined above.

According to another of its aspects, the invention also relates to the microcapsules per se as defined above.

The invention will be explained more clearly by means of the examples hereinafter, given solely by way of illustration and which make it possible to fully understand the invention and to reveal its preparation and/or implementation variants and also its various advantages.

### DESCRIPTION OF THE FIGURES

FIG. 1 represents the fraction by mass of omeprazole released as a function of time at pH=6.8 and 37° C. in a type II dissolutest for a gelatin capsule of sustained-release microcapsules, tested at a dose of 80 mg and at 100 rpm, and prepared according to example 2.

FIG. 2 represents the fraction by mass of omeprazole released as a function of time at pH=6.8 and 37° C. in a type II dissolutest for a gelatin capsule of sustained-release microcapsules, tested at a dose of 40 mg and at 100 rpm, and prepared according to example 3.

FIG. 3 represents the fraction by mass of omeprazole released as a function of time at pH=6.8 and 37° C. in a type II dissolutest for sustained-release microcapsules, tested at doses of 10 mg (diamonds), 40 mg (squares) and 80 mg (triangles) and at 100 rpm, and prepared according to example 5.

FIG. 4 represents the fraction by mass of omeprazole released as a function of time at pH=6.8 and 37° C. in a type II dissolutest for a gelatin capsule of sustained-release microcapsules, tested at a dose of 40 mg and at 120 rpm, and prepared according to example 7.

FIG. 5 represents the fraction by mass of omeprazole released as a function of time at pH=6.8 and 37° C. in a type II dissolutest at the dose of 80 mg and at 150 rpm, for sustained-release microcapsules (crosses x) prepared according to example 7, and for a sustained-release tablet (asterisks *) containing them, prepared according to example 8.

FIG. 6 represents the fraction by mass of omeprazole released as a function of time at pH=6.8 and 37° C. in a type II dissolutest at the dose of 80 mg and at 150 rpm, for sustained-release microcapsules (open squares) prepared according to example 7, and for a sustained-release tablet (solid squares) containing them, prepared according to example 9.

FIG. 7 represents the fraction by mass of omeprazole released as a function of time at pH=6.8 and 37° C. in a type II dissolutest at the dose of 80 mg and at 150 rpm, for sustained-release microcapsules (open triangles) prepared according to example 7, and for a sustained-release tablet (solid triangles) containing them, prepared according to example 10.

FIG. 8 represents the fraction by mass of omeprazole released as a function of time at pH=6.8 and 37° C. in a type II dissolutest at the dose of 80 mg and at 150 rpm, for sustained-release microcapsules (open diamonds) prepared according to example 7, and for a sustained-release tablet (solid diamonds) containing them, prepared according to example 11.

FIG. 9 represents the evolution of the concentration (ng/ml) of omeprazole in the plasma over time after the intake, at day No. 1 after once-daily administration of P1 (solid triangles) and F1 (solid diamonds).

FIG. 10 represents the evolution of the concentration (ng/ml) of omeprazole in the plasma over time after the intake, at day No. 5 after once-daily administration of P1 (open triangles) and F1 (open diamonds).

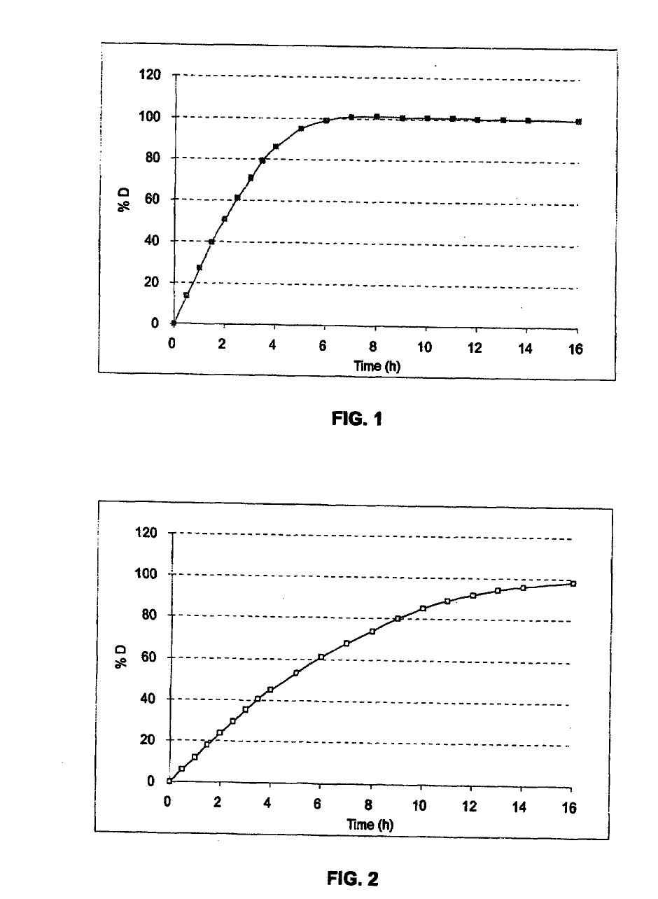

In FIGS. 1 to 10, the x-axis represents the time expressed in hours.

### EXAMPLES

### Example 1

### Omeprazole Granule

700 g of omeprazole and 100 g of hydroxypropylcellulose (Klucel EF®/Aqualon) are dispersed in 3000 g of isopropanol. The suspension is sprayed onto 200 g of neutral microspheres (Asahi-Kasei) in a Glatt GPCG1 spray-coater.

The granule obtained has an omeprazole concentration of 70%.

### Example 2

### Microcapsules with Sustained Release of Omeprazole

50 g of ethylcellulose (Ethocel 20 Premium®/Dow), 20 g of povidone (Plasdone K29/32®/ISP), 20 g of poloxamer 188 (Lutrol F-68®/BASF) and 10 g of castor oil are dispersed in a mixture composed of 60% of isopropanol and 40% of acetone. This solution is sprayed onto 900 g of omeprazole granules (prepared in example 1).

The microcapsules obtained are then placed in a gelatin capsule of size 3. The dose of omeprazole per gelatin capsule was fixed in this test at 80 mg, i.e. 127 mg of microcapsules). This gelatin capsule constitutes the final form of the medicament.

The gelatin capsule containing the microcapsules was tested in a type II dissolutest in accordance with the Pharmacopoeia at 37° C. and with stirring at 100 rpm, at pH 6.8 (0.05M KH₂PO₄/NaOH). See FIG. 1.

### Example 3

### Microcapsules with Sustained Release of Omeprazole

100 g of ethylcellulose (Ethocel 20 Premium®/Dow), 40 g of povidone (Plasdone K29/32®/ISP), 40 g of poloxamer 188 (Lutrol F-68®/BASF) and 20 g of castor oil are dispersed in a mixture composed of 60% of isopropanol and 40% of acetone. This solution is spread onto 800 g of omeprazole granules (prepared in example 1).

The microcapsules obtained are then placed in a gelatin capsule of size 3. The dose of omeprazole per gelatin capsule was fixed in this test at 40 mg, i.e. 71.4 mg of microcapsules). This gelatin capsule constitutes the final form of the medicament.

The gelatin capsule containing the microcapsules was tested in a type II dissolutest in accordance with the Pharmacopoeia at 37° C. and with stirring at 100 rpm, at pH 6.8 (0.05M KH₂PO₄/NaOH). See FIG. 2.

### Example 4

### Omeprazole Granule

900 g of omeprazole and 100 g of hydroxypropylcellulose (Klucel EF®/Aqualon) are dispersed in 2333 g of water. The suspension is sprayed onto 250 g of neutral microspheres (Celphere SCP100F/Asahi-Kasei) in a Glatt GPCG1 spray-coater.

The granule obtained has an omeprazole concentration of 72%.

### Example 5

### Microcapsules with Sustained Release of Omeprazole

8.89 g of ethylcellulose (Ethocel 20 Premium®/Dow), 8.89 g of copolymer of alkyl (meth)acrylate and of trimethylammonioethyl methacrylate chloride (Eudragit RL100®/Degussa Rohm Pharma Polymers), 6.94 g of povidone (Plasdone K29/32®/ISP), 1.94 g of polyoxy-ethylenated hydrogenated castor oil comprising 40 EO (Cremophor RH40®/BASF) and 1.11 g of castor oil are dispersed in a mixture composed of 90% of isopropanol and 10% of water. This solution is sprayed onto 250 g of omeprazole granules (prepared in example 4).

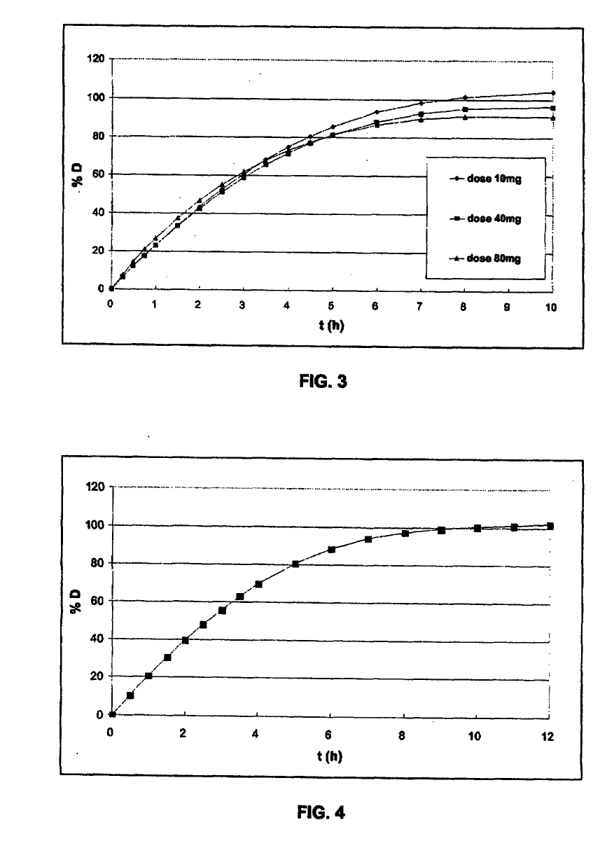

123.4 mg of microcapsules, i.e. 80 mg of omeprazole, were tested in a type II dissolutest in accordance with the Pharmacopoeia at 37° C. and with stirring at 100 rpm, at pH=6.8 (0.05M KH₂PO₄/NaOH). See FIG. 3.

61.7 mg of microcapsules, i.e. 40 mg of omeprazole, were tested in a type II dissolutest in accordance with the Pharmacopoeia at 37° C. and with stirring at 100 rpm, at pH=6.8 (0.05M KH₂PO₄/NaOH). See FIG. 3.

15.4 mg of microcapsules, i.e. 10 mg of omeprazole, were tested in a type II dissolutest in accordance with the Pharmacopoeia at 37° C. and with stirring at 100 rpm, at pH=6.8 (0.05M KH₂PO₄/NaOH). See FIG. 3.

### Example 6

### Omeprazole Granule

1355.2 g of omeprazole, 140.8 g of hydroxypropyl-cellulose (Klucel EF®/Aqualon) 88.0 g of poloxamer 188 (Lutrol F-68®/BASF) and 176.0 g of magnesium hydroxide (Magnesia 725®/Magnesia), incorporated as internal buffering agent, are dispersed in 4107.0 g of water. The suspension is sprayed onto 440.0 g of neutral microspheres (Asahi-Kasei) in a Glatt GPCG1 spray-coater.

The granule obtained has an omeprazole concentration of 61.6%.

### Example 7

### Microcapsules with Sustained Release of Omeprazole

35.0 g of ethylcellulose (Ethocel 20 Premium®/Dow), 17.5 g of povidone (Plasdone K29/32®/ISP), 14.0 g of poloxamer 188 (Lutrol F-68®/BASF) and 3.5 g of castor oil are dispersed in a mixture composed of 70% of ethanol and 30% of water. This solution is sprayed onto 630 g of omeprazole granules (prepared in example 6).

The microcapsules obtained are then placed in a gelatin capsule, with 138.9 g of Destab Ultra 250S® (Particle Dynamic Inc.) corresponding to 125.0 mg of calcium carbonate and 13.9 g of pregelatinized starch, 125.0 mg of magnesium oxide, 3.4 mg of colloidal anhydrous silica (Aerosil 200®/Degussa) and 1.7 mg of magnesium stearate. The dose of omeprazole per gelatin capsule was fixed in this test at 840 mg, i.e. 12472.2 mg of microcapsules. This gelatin capsule constitutes the final form of the medicament.

The gelatin capsule containing the microcapsules was tested in a type II dissolutest in accordance with the Pharmacopoeia at 37° C. and with stirring at 120 rpm, at pH=6.8 (0.05M KH₂PO₄/NaOH). See FIG. 4.

### Example 8

### Tablets with Sustained Release of Omeprazole

144.1 g of sustained-release microcapsules prepared according to example 7 and corresponding to 80 g of omeprazole, 200 g of magnesium oxide (Scora), 9.9 g of crospovidone (Polyplasdone XL10®/ISP), 34.8 g of mannitol (Pearlitol SD200®/Roquette), 100.7 g of microcrystalline cellulose (Ceolus KG-802®/Asahi Kasei) and 7.5 g of glyceryl behenate (Compritol 888 Ato®/Gattefosse) are mixed in a Rohen wheel mixer. The mixture obtained is used to prepare 1000 tablets, each containing 80 mg of omeprazole, using an alternate tablet press (model EK0-Korsh). The tablets obtained have a hardness of between 100 and 150 N.

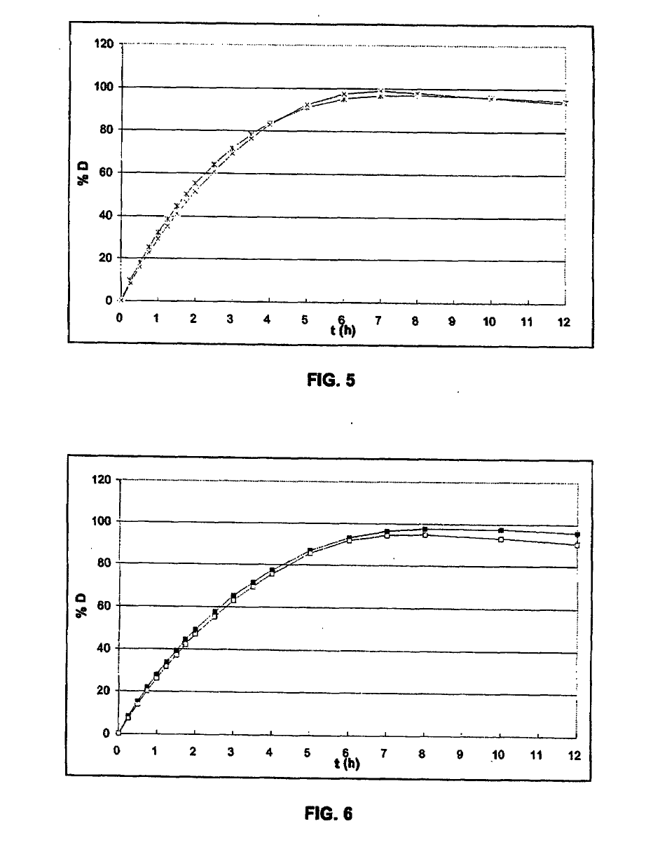

The tablets of 497 mg, i.e. 80 mg of omeprazole, were tested in a type II dissolutest in accordance with the Pharmacopoeia at 37° C. and with stirring at 150 rpm, at pH=6.8 (0.05M KH₂PO₄/NaOH). See FIG. 5.

### Example 9

### Tablets with Sustained Release of Omeprazole

144.1 g of sustained-release microcapsules prepared according to example 7 and corresponding to 80 g of omeprazole, 300 g of magnesium oxide (Scora), 12.2 g of crospovidone (Polyplasdone XL10®/ISP), 21.3 g of mannitol (Pearlitol SD200®/Roquette), 121.5 g of microcrystalline cellulose (Ceolus KG-802®/Asahi Kasei) and 9.1 g of glyceryl behenate (Compritol 888 Ato®/Gattefosse) are mixed in a Rohen wheel mixer. The mixture obtained is used to prepare 1000 tablets, each containing 80 mg of omeprazole, using an alternate tablet press (model EK0-Korsh). The tablets obtained have a hardness of between 100 and 150 N.

A tablet of 608.2 mg, i.e. 80 mg of omeprazole, was tested in a type II dissolutest in accordance with the Pharmacopoeia at 37° C. and with stirring at 150 rpm, at pH=6.8 (0.05M KH₂PO₄/NaOH). See FIG. 6.

### Example 10

### Tablets with Sustained Release of Omeprazole

144.1 g of sustained-release microcapsules prepared according to example 7 and corresponding to 80 g of omeprazole, 200 g of magnesium oxide (Scora), 166.7 g of Destab Ultra 250S® (Particle Dynamic Inc.) corresponding to 150.0 mg of calcium carbonate and 16.7 g of pregelatinized starch, 11.8 g of crospovidone (Polyplasdone XL10®/ISP), 59.3 g of microcrystalline cellulose (Ceolus KG-802®/Asahi Kasei) and 8.9 g of glyceryl behenate (Compritol 888 Ato®/Gattefosse) are mixed in a Rohen wheel mixer. The mixture obtained is used to prepare 1000 tablets, each containing 80 mg of omeprazole, using an alternate tablet press (model EK0-Korsh). The tablets obtained have a hardness of between 100 and 150 N.

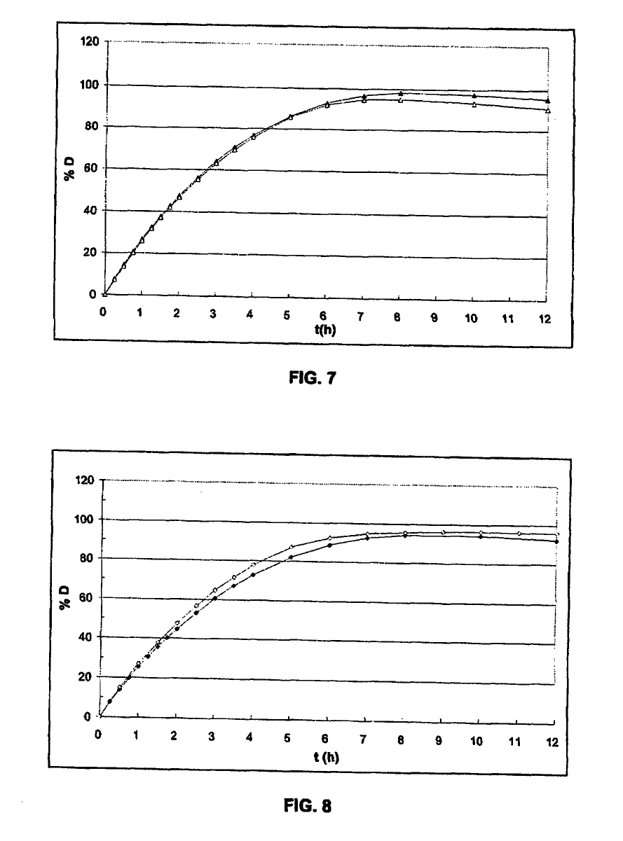

The tablets of 590.8 mg, i.e. 80 mg of omeprazole, were tested in a type II dissolutest in accordance with the Pharmacopoeia at 37° C. and with stirring at 150 rpm, at pH=6.8 (0.05M KH₂PO₄/NaOH). See FIG. 7.

### Example 11

### Tablets with Sustained Release of omeprazole

144.1 g of sustained-release microcapsules prepared according to example 7 and corresponding to 80 g of omeprazole, 250 g of magnesium oxide (Scora), 277.8 g of Destab Ultra 250S® (Particle Dynamic Inc.) corresponding to 250.0 mg of calcium carbonate and 27.8 g of pregelatinized starch, 15.6 g of crospovidone (Polyplasdone XL10®/ISP), 80.0 g of microcrystalline cellulose (Ceolus KG-802®/Asahi Kasei) and 11.7 g of glyceryl behenate (Compritol 888 Ato®/Gattefosse) are mixed in a Rohen wheel mixer. The mixture obtained is used to prepare 1000 tablets, each containing 80 mg of omeprazole, using an alternate tablet press (model EK0-Korsh). The tablets obtained have a hardness of between 100 and 150 N.

The tablets of 779.2 mg, i.e. 80 mg of omeprazole, were tested in a type II dissolutest in accordance with the Pharmacopoeia at 37° C. and with stirring at 150 rpm, at pH=6.8 (0.05M KH₂PO₄/NaOH). See FIG. 8.

### Example 12

### In Vivo Data

Enteric Oral Medicament with Reference* Immediate Release of the S(−) Enantiomer of Omeprazole

P1: Gelatin capsules containing microcapsules of S(−)-omeprazole film-coated with an enteric membrane —Inexium®-dose 40 mg.

Nonenteric Oral Medicament with Modified Release of Racemic Omeprazole According to the Invention

F1: 2 gelatin capsules of omeprazole of example 7-omeprazole dose 80 mg.

### Description of the Trial

The reference* enteric medicament P1 or the nonenteric oral medicament with modified release of omeprazole according to the invention F1 is administered once-daily, after 10 h of fasting and before breakfast, for five days, to 28 normal volunteers in the course of a crosstrial. The plasma concentrations of omeprazole are measured at the times: 0-0.5-0.75-1-1.5-2-3-4-6-8-10-12-14-16-20-24 h post-administration, on days No. 1 and No. 5, by means of an LC-MS method. The gastric pH is measured by means of a Digitrapper® pH 100 probe every 4 seconds over 24 hours post-administration.

### Pharmacokinetic Results

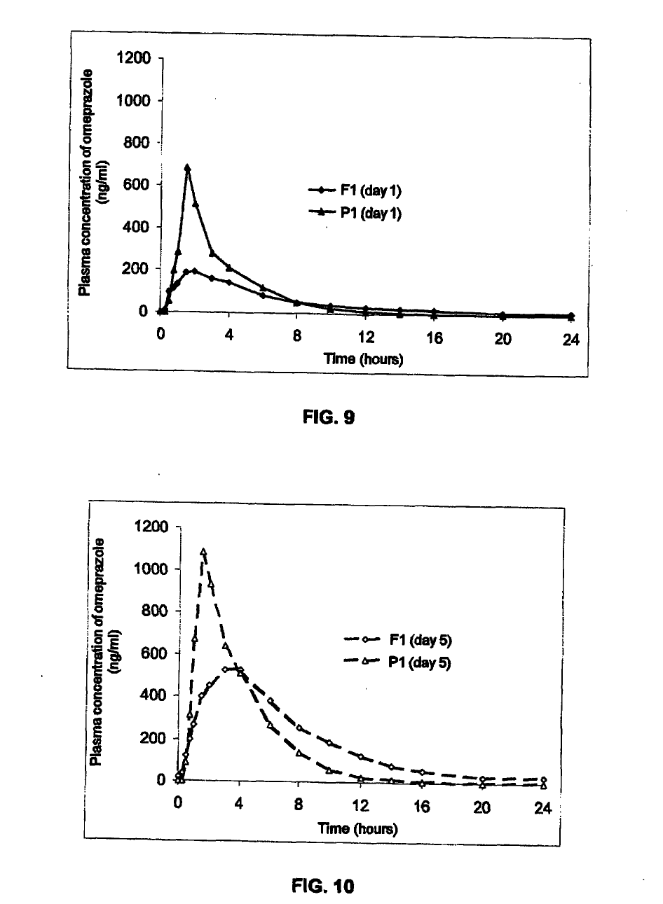

The pharmacokinetic profiles of the omeprazole as a function of time after dose intake on days No. 1 and No. 5 are described in FIG. 9 and in FIG. 10, respectively.

The mean pharmacokinetic parameters (Cmax, Tmax, AUC₀₋₂₄ₕ, C12h, Cmax/C12h ratio) and their standard deviation for the reference* enteric medicament P1 and the nonenteric oral medicament with modified release of omeprazole according to the invention F1 are given in table 1 below:

TABLE 1

| | Cmax | Tmax | AUC_{0-24 h} | | |
|---|---|---|---|---|---|
| Treatment | (ng/ml) | (h) | (ng/ml × h) | C12 h (ng/ml) | Cmax/C12 h |
| | Day No. 1 | | | | |
| P1 | 615 ± 547 | 1.5 (0.75-6.0) | 1392 ± 1141 | 9 ± 20 | 94.8 |
| F1 | 188 ± 122 | 2 (0.75-4.0) | 901 ± 973 | 29 ± 50 | 7.7 |
| | Day No. 5 | | | | |
| P1 | 1160 ± 499 | 1.5 (0.75-6.00) | 3555 ± 1777 | 24 ± 46 | 55.0 |
| F1 | 501 ± 260 | 3.0 (1.5-6.0) | 3455 ± 2314 | 125 ± 112 | 4.7 |

The medicament F1 according to the invention improves the Cmax/C12h parameter by a factor of approximately 12 for days No. 1 and No. 5 compared with the reference* form P1.

### Pharmacodynamic Results

The means, obtained on day No. 1, of the:
time over 24 h (as % and in h) for which the gastric pH is greater than 4 (T>pH 4) and 5 (T>pH 5), of the
mean gastric pH over 24 h (mean pH 0-24 h), and of the
mean gastric pH determined overnight (mean pH overnight 16-20 h)
   are reported in table 2 below:

TABLE 2

| | Day No. 1 | | | |
|---|---|---|---|---|
| | | | | mean pH |
| | T > pH 4 | T > pH 5 | mean pH | overnight |
| Treatment | % h | % h | 0-24 h | 16-20 h |
| P1 | 41.1 ± 9.9 | 24.0 ± 5.8 | 3.17 ± 0.73 | 1.68 ± 0.98 |
| | 16.9 | 12.5 | | |
| F1 | 43.6 ± 10.5 | 26.2 ± 6.3 | 3.30 ± 1.01 | 2.49 ± 1.53 |
| | 20.8 | 16.9 | | |

The medicament F1 according to the invention clearly increases the mean pH over 24 h compared with the reference* enteric oral medicament P1.

It can also be noted that the mean pH overnight is clearly increased by the formulation F1 according to the invention compared with the reference* enteric form P1. The formulation F1 according to the invention thus makes it possible to obtain a gastric pH overnight (between 16 and 20 h), of day No. 1, equal to 2.49, whereas the mean pH overnight is 1.68 with the reference* form P1.

The means, obtained on day No. 5, of the:
time over 24 h (as % and in h) for which the gastric pH is greater than 4 (T>pH 4) and 5 (T>pH 5), of the
mean gastric pH over 24 h (mean pH 0-24 h), and of the
mean gastric pH determined overnight (mean pH overnight 16-20 h)
   are reported in table 3 below:

TABLE 3

| | Day No. 5 | | | |
|---|---|---|---|---|
| | | | | mean pH |
| | T > pH 4 | T > pH 5 | mean pH | overnight |
| Treatment | % h | % h | 0-24 h | 16-20 h |
| P1 | 61.48 ± 14.7 | 43.33 ± 10.4 | 4.14 ± 0.53 | 1.93 ± 0.94 |
| | 10.94 | 10.4 | | |
| F1 | 73.94 ± 17.7 | 54.79 ± 13.1 | 4.36 ± 1.55 | 3.44 ± 1.21 |
| | 13.78 | 16.82 | | |

The medicament F1 according to the invention clearly increases the mean pH over 24 h and the time during which the gastric pH is greater than 4 (T>pH 4) compared with the reference* enteric oral medicament P1.

It can also be noted that the mean pH overnight is clearly increased by the formulation F1 according to the invention compared with the reference* enteric form P1. The formulation F1 according to the invention thus makes it possible to obtain a gastric pH overnight (between 16 and 20 h), of day No. 5, equal to 3.44, whereas the mean pH overnight is 1.93 with the reference* form P1.

1. A PPI-based oral medicament allowing modified release of this PPI, characterized in that it is designed such that, after its ingestion as a once-daily dose, it makes it possible to maintain, from the first day of treatment onward, an average gastric pH, between 0 and 24 h, that is greater than or equal to the average gastric pH, between 0 and 24 h, obtained with a reference* immediate-release enteric oral medicament administered under the same conditions.

2. A PPI-based oral medicament allowing modified release of this PPI, optionally as claimed in claim 1, characterized in that it is designed such that, after its ingestion, the release of the PPI begins in the stomach and that, when it is administered as a once-daily dose in the morning, it makes it possible to maintain, from the fifth day of treatment onward, the average gastric pH, between 16 hours and 20 hours after the dose has been taken, at greater, preferably greater by at least 0.5 pH unit, and even better still greater by at least 1 pH unit, than the average gastric pH, between 16 hours and 20 hours after the dose has been taken, obtained with a reference* immediate-release enteric oral medicament administered under the same conditions.

3. A PPI-based oral medicament allowing modified release of this PPI, optionally as claimed in claim 1 or 2, characterized in that it is designed such that, after its ingestion as a once-daily dose, it makes it possible to maintain, from the first day of treatment onward, a gastric pH that is greater than or equal to the gastric pH obtained with a reference* immediate-release enteric oral medicament administered under the same conditions, for at least 16 h, preferably at least 20 h, and even more preferably at least 22 h.

4. A PPI-based oral medicament allowing modified release of this PPI, optionally as claimed in claim 1, 2 or 3, characterized in that it is designed such that, after its ingestion as a once-daily dose, it makes it possible to maintain, from the fifth day of treatment onward, a gastric pH that is greater than or equal to the gastric pH obtained with a reference* immediate-release enteric oral medicament administered under the same conditions, for at least 13 h, preferably at least 16 h, and even more preferably at least 20 h.

5. The medicament as claimed in at least one of the preceding claims, characterized in that it is of reservoir type.

6. The medicament as claimed in at least one of the preceding claims, characterized in that it comprises a plurality of microcapsules with modified release of PPI, these microcapsules individually comprising at least one microparticle containing PPI and coated with at least one coating which allows the modified release of the PPI.

7. The medicament as claimed in at least one of the preceding claims, characterized in that it is designed such that, after its ingestion, the release of the PPI begins in the stomach, and in that, when it is administered as a once-daily dose, it makes it possible to maintain, from the first day of treatment onward, the gastric pH at a value greater than or equal to 4.0, for a period of time D greater than or equal to the period of time D* during which the pH is maintained at a value greater than or equal to 4.0 with a reference* immediate-release enteric oral medicament administered under the same conditions, D preferably being greater than or equal to D* by at least 5% (% relative to D), more preferably by at least 10%, and even more preferably by at least 20%.

8. The medicament as claimed in at least one of claims 1 to 6, characterized in that it is designed such that, after its ingestion, the release of the PPI begins in the stomach, and in that, when it is administered as a once-daily dose, it makes it possible to maintain, from the fifth day of treatment onward, the gastric pH at a value greater than or equal to 4.0, for a period of time D greater than or equal to the period of time D* during which the pH is maintained at a value greater than or equal to 4.0 with a reference* immediate-release enteric oral medicament administered under the same conditions, D preferably being greater than or equal to D* by at least 5% (% relative to D), more preferably by at least 10%, and even more preferably by at least 20%.

9. The medicament as claimed in at least one of the preceding claims, characterized in that it is nonenteric or that the coating of the PPI microcapsules optionally included in this medicament is nonenteric.

10. The medicament as claimed in at least one of the preceding claims, characterized in that it is designed such that, when it is administered as a once-daily dose, it makes it possible to obtain, after the dose has been taken, a plasma profile defined as follows:
Cmax/C12h≦Cmax*/C12h*
preferably
1.5×Cmax/C12h≦Cmax*/C12h*
and even more preferably
2.0×Cmax/C12h≦Cmax*/C12h*
with
C12h representing the mean plasma concentration of PPI 12 h after the dose has been taken,
C12h* representing the mean plasma concentration of PPI obtained under the same conditions as C12h, with a reference* immediate-release enteric oral medicament containing the same dose of PPI,
Cmax representing the mean maximum plasma concentration of PPI after the dose has been taken,
Cmax* representing the mean maximum plasma concentration of PPI, obtained under the same conditions as Cmax, with a reference* immediate-release enteric oral medicament containing the same dose of PPI.

11. The medicament as claimed in at least one of the preceding claims, characterized in that the PPI microcapsules have an in vitro release profile in potassium dihydrogen phosphate/sodium hydroxide (0.05M) buffer medium, at pH 6.8, such that:
70% of the PPI is released in a time of between 1 and 10 hours, preferably between 2 and 8 hours, and even more preferably between 2 and 6 hours, and
40% of the PPI is released in a time of between 0.5 and 5 hours, preferably between 1 and 4 hours, and even more preferably between 1 and 3 hours.

12. The medicament as claimed in claim 11, characterized in that the PPI microcapsules have an in vitro release profile in potassium dihydrogen phosphate/sodium hydroxide (0.05M) buffer medium, at pH 6.8, such that, for any value of the time t of between 2 h and t(70%), preferably for any value of the time t of between 1 h and t(70%), the percentage of dissolved (released) PPI is greater than or equal to 35t/t(70%).

13. The medicament as claimed in at least one of the preceding claims, characterized in that it contains at least one external buffering agent, which preferably comprises at least one weakly or strongly basic, pharmaceutically acceptable compound.

14. The medicament as claimed in claim 13, characterized in that the external buffering agent is chosen from the group comprising the following products: amino acids and their salts, sodium salts, potassium salts, calcium salts, magnesium salts, aluminum salts, these salts preferably being selected from the following salts: hydroxides, oxides, lactates, gluconates, carbonates, sesquicarbonates, bicarbonates, silicates, phosphates, glycerophosphates, pyro-phosphates, polyphosphates, chlorides, and mixtures thereof.

15. The medicament as claimed in claim 13 or 14, characterized in that it contains between 0 and 100 mEq, preferably between 2 and 40 mEq, of external buffering agent(s).

16. The medicament as claimed in at least one of claims 13 to 15, characterized in that the external buffering agent comprises calcium carbonate.

17. The medicament as claimed in claim 16, characterized in that the calcium carbonate is present in a proportion of 2 to 15 mEq, preferably 5 to 10 mEq.

18. The medicament as claimed in at least one of claims 13 to 17, characterized in that the external buffering agent comprises magnesium oxide.

19. The medicament as claimed in at least one of claims 13 to 18, characterized in that the external buffering agent selected comprises between 5 mEq and 35 mEq, preferably between 5 mEq and 25 mEq, of magnesium oxide.

20. The medicament as claimed in at least one of claims 13 to 19, characterized in that the external buffering agent comprises from 3 to 7 mEq of calcium carbonate and an amount of magnesium oxide such that the magnesium oxide/calcium carbonate ratio, in milliequivalents, is between 1.5 and 5.

21. The medicament as claimed in at least one of claims 13 to 20, characterized in that the external buffering agent selected comprises magnesium hydroxide.

22. The medicament as claimed in at least one of claims 13 to 21, characterized in that the external buffering agent comprises between 5 mEq and 30 mEq, preferably between 5 and 20 mEq, of magnesium hydroxide.

23. The medicament as claimed in at least one of claims 13 to 22, characterized in that the external buffering agent comprises from 3 to 7 mEq of calcium carbonate and an amount of magnesium hydroxide such that the magnesium hydroxide/calcium carbonate ratio, in milliequivalents, is between 1.5 and 5.

24. The medicament as claimed in at least one of claims 13 to 23, characterized in that the external buffering agent is immediate-release.

25. The medicament as claimed in at least one of the preceding claims, characterized in that the PPI microcapsules contain at least one internal buffering agent.

26. The medicament as claimed in claim 25, characterized in that the PPI microcapsules contain at least one internal buffering agent comprising magnesium hydroxide.

27. The medicament as claimed in at least one of the preceding claims, characterized in that the coating of the PPI microcapsules comprises at least one layer which controls the modified release of the PPI and the composition of which is the following:
A. at least one film-forming (co)polymer (A) which is insoluble in the fluids of the gastrointestinal tract;
B. optionally at least one hydrophilic film-forming (co)polymer (B) which
is insoluble in the fluids of the gastrointestinal tract,
bears groups which are ionized in the fluids of the gastrointestinal tract;
C. at least one (co)polymer (C) which is soluble in the fluids of the gastrointestinal tract;
D. at least one plasticizer (D);
E. optionally at least one surfactant and/or lubricant (E).

28. The medicament as claimed in claim 27, characterized in that
(A) is selected from the group of following products:
water-insoluble derivatives of cellulose,
preferably ethylcellulose and/or cellulose acetate,
polyvinyl acetates,
and mixtures thereof;
(B), when it is present, is chosen from water-insoluble charged acrylic polymers, preferably from (co)polymers of an ester of acrylic and/or methacrylic acid bearing at least one quaternary ammonium group; (B) even more preferably comprising at least one copolymer of alkyl (meth)acrylate and of trimethylammonioethyl meth-acrylate chloride;
(C) is chosen from:
nitrogenous (co)polymers, preferably from the group comprising polyacrylamides, poly-N-vinylamides, polyvinylpyrrolidones (PVPs) and poly-N-vinyllactams;
water-soluble derivatives of cellulose,
polyvinyl alcohols (PVAs),
polyoxyethylenes (POEs),
polyethylene glycols (PEGs),
hydrocolloids, such as xanthan gums, guar gums, pectins, carob gum, carrageenans, gelatin, agar agar, modified or unmodified starches, dextrins or alginates,
and mixtures thereof,
polyvinylpyrrolidone, polyoxyethylenes, polyethylene glycols and hydroxypropylcellulose being particularly preferred;
(D) is chosen from the group comprising:
cetyl alcohol esters,
glycerol and esters thereof, preferably from the following subgroup: acetyl glycerides, glyceryl monostearate, glyceryl triacetate (triacetin), glyceryl tributyrate,
phthalates, preferably from the following subgroup: dibutyl phthalate, diethyl phthalate, dimethyl phthalate, dioctyl phthalate,
citrates, preferably from the following subgroup: acetyltributyl citrate, acetyltriethyl citrate, tributyl citrate, triethyl citrate,
sebacates, preferably from the following subgroup: diethyl sebacate, dibutyl sebacate,
adipates,
azelates,
benzoates,
plant oils,
fumarates, and preferably diethyl fumarate,
malates, preferably diethyl malate,
oxalates, preferably diethyl oxalate,
succinates, preferably dibutyl succinate,
butyrates,
salicylic acid,
malonates, preferably diethyl malonate,
castor oil (the latter being particularly preferred),
and mixtures thereof;
(E) is chosen from the group comprising:
anionic surfactants, preferably from the subgroup of alkali metal salts or alkaline earth metal salts of fatty acids, stearic acid and/or oleic acid being preferred,
and/or nonionic surfactants, preferably from the following subgroup:
polyoxyethylenated oils, preferably polyoxy-ethylenated hydrogenated castor oil,
polyoxyethylene/polyoxypropylene copolymers,
polyoxyethylenated sorbitan esters,
polyoxyethylenated castor oil derivatives,
stearates, preferably calcium stearate, magnesium stearate, aluminum stearate or zinc stearate,
stearyl fumarates, preferably sodium stearyl fumarate,
glyceryl behenates,
and mixtures thereof.

29. The medicament as claimed in claim 27 or 28, characterized in that the composition of the modified-release layer is the following:
A. the film-forming polymer(s) (A) is (are) present in a proportion of 10% to 90%, preferably 20% to 40% by weight on a dry basis, relative to the total mass of the coating composition;
B. the water-insoluble hydrophilic film-forming polymer(s) (B) is (are) present in a proportion of 0% to 90%, preferably 0% to 40% by weight on a dry basis, relative to the total mass of the coating composition;
C. the polymer(s) (C) which is (are) soluble is (are) present in a proportion of 2% to 25%, preferably 5% to 15% by weight on a dry basis, relative to the total mass of the coating composition;
D. at least one plasticizer (D) is present in a proportion of 2% to 20%, preferably 4% to 15% by weight on a dry basis, relative to the total mass of the coating composition;
E. the optional surfactant(s) and/or lubricant(s) (E) is (are) present in a proportion of 2% to 20%, preferably 4% to 15% by weight on a dry basis, relative to the total mass of the coating composition.

30. The medicament as claimed in at least one of the preceding claims, characterized in that the diameter of the microcapsules is less than or equal to 1000 μm, preferably between 5 and 800 μm, and even more preferably between 100 and 600 μm.

31. The medicament as claimed in at least one of the preceding claims, characterized in that the proportion of PPI in the microcapsules (expressed as % by weight on a dry basis, relative to the total mass of the microcapsules) is between 5 and 95, preferably between 10 and 85, and even more preferably between 20 and 70.

32. The medicament as claimed in claim 27 or 28, comprising PPI microcapsules in which the composition of the modified-release layer is the following:
A. the film-forming polymer(s) (A) is (are) present in a proportion of 40% to 55%, preferably 45% to 55% by weight on a dry basis, relative to the total mass of the coating composition;
C. the soluble polymer(s) (C) is (are) present in a proportion of 15% to 30%, preferably 20% to 30% by weight on a dry basis, relative to the total mass of the coating composition;
D. at least one plasticizer (D) is (are) present in a proportion of 3% to 10%, preferably 3% to 7% by weight on a dry basis, relative to the total mass of the coating composition;
E. the optional surfactant(s) and/or lubricant(s) (E) is (are) present in a proportion of 10% to 30%, preferably 15% to 25% by weight on a dry basis, relative to the total mass of the coating composition.

33. The medicament as claimed in claim 32, in which:
(A) is selected from the group of following products: water-insoluble derivatives of cellulose, preferably ethylcellulose and/or cellulose acetate;
(C) is chosen from polyvinylpyrrolidones (PVPs) and water-soluble derivatives of cellulose, such as hydroxypropylcellulose; PVPs being preferred;
(D) is castor oil;
(E) is chosen from: polyoxyethylene/polyoxypropylene copolymers, preferably polyoxyethylene/polyoxypropylene block terpolymers.

34. The medicament as claimed in claim 32 or 33, in which the PPI is omeprazole.

35. The medicament as claimed in claim 34, characterized in that the volume-average diameter of the omeprazole microcapsules is between 100 and 500 μm, preferably between 100 and 400 μm, and more preferably between 100 and 300 μm.

36. The medicament as claimed in at least one of claims 32 to 35, in which the PPI microcapsules are omeprazole microcapsules, and in which said microcapsules have an in vitro release profile in potassium dihydrogen phosphate/sodium hydroxide (0.05M) buffer medium, at pH 6.8, such that:
70% of the omeprazole is released in a time of between 2 h and 8 h, preferably between 2 h and 5 h,
40% of the omeprazole is released in a time of between 1 h and 4 h, preferably between 1 h and 3 h,
at least 70% of the omeprazole, preferably at least 90% of the omeprazole, is released in 10 h.

37. The medicament as claimed in any one of claims 32 to 36, in which, in the omeprazole microcapsules, the modified-release layer represents from 2% to 25% by weight, preferably from 5% to 20% by weight, and more preferably from 5% to 15% by weight, relative to the total weight of the omeprazole microcapsules.

38. The medicament as claimed in at least one of the preceding claims, characterized in that it also comprises at least one H2 receptor antagonist, preferably selected from the group comprising the following active ingredients: cimetidine, ranitidine, nizatidine, famotidine, pharmaceutically acceptable salts thereof, isomers thereof and salts of an isomer thereof, and also any mixture of these various active ingredients.

39. The medicament as claimed in at least one of the preceding claims, characterized in that it is formed by various populations of microunits, these populations differing from one another at least by virtue of the nature of the active ingredient(s) other than the PPI that is (are) contained in these microunits and/or by virtue of the amount of PPI or of other optional active ingredient(s) that they contain and/or by virtue of the composition of the coating and/or by virtue of the fact that they are modified-release or immediate-release.

40. The medicament as claimed in at least one of the preceding claims, characterized in that it is in the form of a once-daily oral dose comprising from 1 mg to 500 mg of PPI.

41. The medicament as claimed in one of the preceding claims, characterized in that it is in the form of a sachet of powder, a multidose suspension reconstituted from water and powder, a tablet or a gell capsule.

42. The medicament as claimed in any one of the preceding claims, characterized in that it is in tablet form, the tablet having an in vitro release profile in potassium dihydrogen phosphate/sodium hydroxide (0.05M) buffer medium, at pH 6.8, similar to that of the microcapsules of said tablet, according to the similarity factor f2.

43. The medicament as claimed in claim 42, in which the tablet contains, in addition to a plurality of microcapsules with modified release of PPI:
from 5 to 25 mEq of external buffering agent, preferably from 10 to 20 mEq of external buffering agent,
compression excipients in an amount such that the total mass of the tablet does not exceed 1000 mg, preferably 800 mg, and more preferably 600 mg.

44. The medicament as claimed in claim 43, in which the external buffering agent is chosen from calcium carbonate, magnesium oxide and mixtures thereof.

45. The medicament as claimed in any one of claims 42 to 44, in which the tablet has a hardness greater, in an increasing order of preference, than 80N, 100N, 120N, and preferably less than 300N, or better still less than 200N.

46. The microcapsules as defined in at least one of claims 6, 9, 11, 12 and 25 to 37.
